(19) United States
(12) Patent Application Publication
(10) Pub. No.: US 2018/0279608 A1
(43) Pub. Date: Oct. 4, 2018

(54) SYSTEM FOR PREVENTION OF INFECTIOUS DISEASE

(71) Applicants: Ilan Yehoshua KEREN, Alonei Abba (IL); ALONIM BREEDERS FARM LTD, Kibbutz Alonim (IL)

(72) Inventors: Ilan Yehoshua Keren, Alonei Abba (IL); Tzuriel HANUNI, Kibbutz Alonim (IL)

(73) Assignees: ALONIM BREEDERS FARM LTD., Kibbutz Alonim (IL); Ilan Yehoshua KEREN, Alonei Abba (IL)

(21) Appl. No.: 15/776,951

(22) PCT Filed: Nov. 8, 2016
(86) PCT No.: PCT/IL2016/051206

§ 371 Date: May 22, 2018

Related U.S. Application Data
(60) Provisional application No. 62/260,853, filed on Nov. 30, 2015.

(51) Int. Cl. A01N 25/34; A01K 1/015; A01M 1/20

(52) U.S. Cl. CPC A01N 25/34; A01K 1/0011; A01M 1/20; A01K 1/015

(57) ABSTRACT

A system for prevention of infectious diseases in a habitat of animals. This system comprises articles consisting at least one antimicrobial additive. Some or all those articles are situated within a bedding of a habitat of animals.

### FIELD OF THE INVENTION

The present invention relates to biosecurity, in particular, to prevention of infectious diseases at habitats of animals.

### BACKGROUND OF THE INVENTION

In 2010, approximately 36.9 billion pounds (16,737,558 tones) of broilers were sold in the US only, for a retail value of $45 billion.

Broiler breeding typically starts with parent stock, purchased from a primary breeder. The parent stock is then sent to a special rearing houses or a starter farms, where it is kept for about 22 weeks at a high level of biosecurity. Biosecurity refers to those measures taken to prevent an introduction and spread of infectious agents to a flock. Such infectious agents significantly reduce the productivity, profitability and long term financial viability of a poultry operation. Factors that may impact on the biosecurity include location, proximity to other production areas, ventilation, water, feed, wildlife and humans.

The internal conditions inside the chicken house, are very significant as well. Wet manure and saturated bedding, along with the massive animal heat generated by so many birds, result in perfect environmental conditions for bacteria development. The existing practices to tackle with the problematic interior environment in coops rely on using disinfectants which may be harmful to humans, animals and the environment. Moreover, the effectiveness of using antibiotics, is limited by the ability of microbes to mutate and acquire resistance rapidly. Studies show that overuse and misuse of antibiotics, kill off the un-resistant bacteria and creates the perfect environment for antibiotic-resistant bacteria to multiply and thrive. Drug-resistant bacteria can hitch a ride out of animal feed lots as well as on workers who handle contaminated animals or meat and even on the meat itself. They can travel through water, soil, and air that come into contact with contaminated animal waste. Resistant bacteria can cause infections that are harder to treat, requiring the use of medicines with greater side effects. They can also lead to longer illnesses and more hospitalizations. A broad coalition of prominent medical and public health groups has warned that the “overuse and misuse of important antibiotics in food animals must end, in order to protect human health.”

In U.S. Ser. No. 12/866,098 Correa describes another approach wherein a chicken house includes a ventilated floor component in the form of flat molded plastic sections with small ventilation openings set side-by-side, through which air and liquid (moisture) can flow. Correa design is aimed to remove the water and moisture from the manure deposited on the floor, to keep it dry so as to reduce ammonia formation, as well as reduce salmonella, E-coli, coccidiosis, and multiple bacterial strain growth. However, the complex structures described by Correa, might cause a substantial increase the investments.

In U.S. Ser. No. 12/866098 Rudi et al. describe an administration of an antimicrobial agent typically with or as part of the birds' normal feed and/or drinking water. In U.S. Pat. No. 6,555,106 Byrd II, et al. describe a method and compositions for controlling food borne enteric bacterial pathogens in poultry populations by providing particles of an expanded matrix material to the locus or vicinity of the animals, particularly during the period of feed removal prior to slaughter. Yet, both Rudi and Byrd seem to disregard the area where the pathogen propagation takes place.

### DESCRIPTION OF THE INVENTION

In a habitat of animals which embodies aspects of the present invention, there are articles which contain at least one antimicrobial additive for protecting animals from infectious diseases. It could be that some or all those articles being, structural parts of a habitat of animals such as walls, ceilings, beams, floors, columns, supporters, grids, lintels frames, panels, dividers, sheds, roofs or fences.

According to an embodiment of the above aspect of the invention, those articles containing antimicrobial additives are situated within the bedding of a habitat of animals. It is an option that the bedding themselves would be solely composed by those articles or in combination with other articles which may contain other additives such as pesticides or insecticides.

According to an aspect of the present invention, the habitat of animals may be a habitat of livestock such as coop, dovecote, house, stable, barn, cote, corral kennel or a pond.

According to an embodiment of the above aspect of the invention, the articles containing antimicrobial additives are fixed or placed on a floor. In some cases it may be desirable that some of the articles containing antimicrobial additives attached to a floor while others are situated within the bedding. In some cases some of the articles are just placed on the floor.

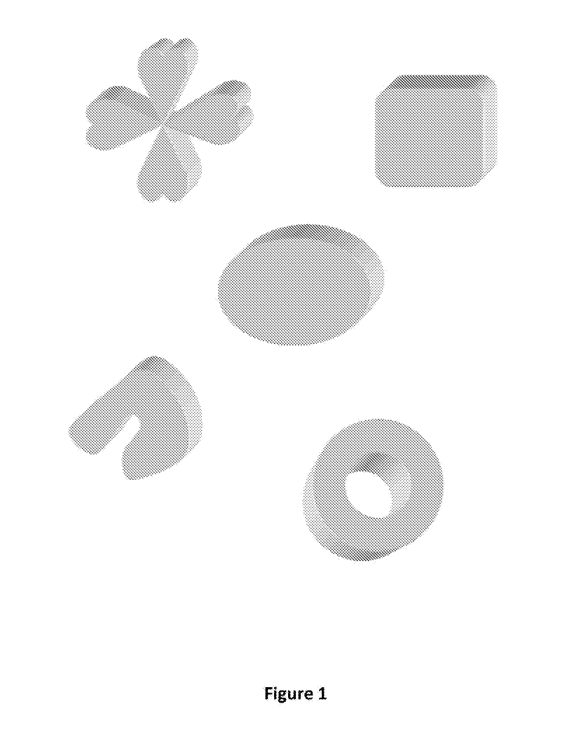

It is preferable that the size of the above articles prevents the animals from swallowing them. Additionally, it is preferable that the material of the articles is such that it repels the animals from biting and eating the articles. Those articles may be in the various shapes such as rounds, spheres, rods or stripes. It is desired that their shape will not contain sharp edges which can injure the animals. Those articles do not need to be in one shape; however this might be advantageous for low cost production. FIG. 1 illustrates possible shapes of antimicrobial articles, embodying the principles of the invention.

According to an aspect embodying the present invention there may be various options for recycling the articles. An example for recycling is sieving the articles from the bedding, and then reusing them in new fresh bedding. Another alternative is using biodegradable or compostable materials for the articles. In the case of using biodegradable or compostable materials for the articles, the required duration for the antimicrobial functionality before biodegradation or compostation, is to be considered.

The articles may be made of plastics consisting antimicrobial additives either leaching or non-leaching. Leaching antimicrobial agents come off the article or substrate in order to exert antimicrobial properties. Non-leaching agents are fixed to the article (usually by covalent bonds), and subsequently do not need to leave the surface.

According to an embodiment of the present invention, equipment and tools may as well, incorporate antimicrobial additives, e.g., constructions parts, working tools, clothing, transportation, vehicles, systems for feeding and water supply, air conditioning and ventilation, piping, nest and salt systems, conveyors, hatcheries, panels, incubators, tray, baskets, doors, inlets, outlets, trolleys, boxes, drinkers, control and measuring instruments, hoppers, silos, tanks, and pans. In some cases, antimicrobial additives may be incorporated only on the surface. For example, when antimicrobial additives are incorporated within coating materials. This may be beneficial when the substrate body is large and it does not make sense to include a big amount of the antimicrobial material in the whole substrate body. Surfaces may become antimicrobial by attaching polymers or polypeptides. Flooring of chicken houses according to principles of the present invention may incorporate antimicrobial additives or merely be covered by antimicrobial surface. Another option is placing carpets or sheets consisting antimicrobial additives instead of treating the floor. Antimicrobial sheets may be in use also to cover the walls or the ceilings. This may be useful when only a provisional protection being required.

Various compounds may be in use as antimicrobial agents. Silver is an example of a compound which is toxic to microorganisms. Silver reacts with the cell wall to inhibit essential functions, including cellular respiration and nutrients uptake. Silver can interfere with the structural integrity of the cell leading to proton leakage, cytoplasmic leakage, or complete rupture of the cell wall, all resulting into cell death. Silver ions can also interfere with enzymatic functions within the cell. The enzymes are responsible for a wide range of intracellular activities, such as breakdown of nutrients, production of proteins and DNA replication.

Researchers believe that elevated copper levels inside a cell generate oxidative stress and hydrogen peroxide. Under these conditions, copper participates in the so-called Fenton-type reaction—a chemical reaction causing oxidative damage to cells. Excess copper causes a decline in the membrane integrity of microbes, leading to leakage of specific essential cell nutrients, such as potassium and glutamate. This leads to desiccation and subsequent cell death. Another consequence of elevated copper levels is a loss-of-function of the protein, and/or breakdown of the protein into nonfunctional portions.

Zinc pyrithione kills microbes by interfering with their proton pump activity. Proton pumps function to open pores in the cell wall, allowing the transport of nutrients into the cell and waste products out of the cell. Without this capability the cell is unable to grow and may thrive and die. The quaternary ammonium compound 3-(Trimethoxysilyl)—propyldimethyloctadecyl ammonium chloride (Si-QAC) has been found to have antimicrobial activity when covalently bonded to a surface. Other quaternary ammonium compounds are known also to have antimicrobial properties such as alkyldimethylbenzylammonium chloride and didecyldimethylammonium chloride.

Silane based antimicrobials act by disrupting the ionic charge of microorganisms cell walls. Those polymers consist of uncharged carbon chains interspersed with positively charged ammonium moieties of the cell wall or cellular membrane, resulting in the rupture of and the death of the microorganism. The antimicrobial mechanism of action for Isothiozolanones is enzyme inhibition. Enzymes regulating cellular respiration, energy production and growth are destroyed. Protein destruction, and subsequent production of free radicals, is the actual cause of cell death.

Insects are prominent disease vectors and they are most difficult to control. Construction materials of a chicken house might be subjected to insects' attacks and it is therefore desired that they will be resistant to such attacks. Those parts do not necessarily provide nutritional value to insects, but an insect, in search of food and shelter may attack them. Insecticides, and more specifically, polymeric insecticides, may be used as a means of a slow release and a long term capability of killing insects that come in contact or within close proximity. Insects repellent can deter insects from nesting and chewing those parts, and by this to damage their required physical properties. Construction materials can undergo surface treatment with insecticides that kill insects that come in contact with the treated surface. These insecticides typically leach out over time out of those treated surfaces. Typically, the reported useful retention time of the insecticide ranges from a month to two or three years at the most. Plastic impregnated with pyrethroid is an example of such a tool for disease vector control. There are other examples of insecticides, some of them are made from natural raw materials. The insecticides may be incorporated together with the antimicrobial additives, or in separate elements and/or articles comprising the chicken house.

While the present invention is described here as relating to chickens and chicken houses, it will be understood by those skilled in the art that the present invention is also applicable to other poultry and poultry houses, such as turkeys, etc. Also, while the invention is also described herein as relating to growing chickens and other poultry, the present invention is also applicable for use in egg laying facilities. Further, it will be understood by those skilled in the art that the present invention is also applicable to other livestock such as ruminants and pigs.

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition, an apparatus, or a method described herein can be combined in a variety of ways without departing from the spirit and scope of the present teachings, whether explicit or implicit herein. For example, where reference is made to a particular structure, that structure can be used in various embodiments of apparatus of the present teachings and/or in methods of the present teachings, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

The use of the singular herein, for example, “a,” “an,” and “the,” includes the plural (and vice versa) unless specifically stated otherwise.

The use of any and all examples, or exemplary language herein, for example, “such as,” “including,” or “for example,” is intended merely to illustrate better the present teachings and does not pose a limitation on the scope of the invention unless claimed. No language in the specification should be construed as indicating any non-claimed element as essential to the practice of the present teachings.

The present teachings encompass embodiments in other specific forms without departing from the spirit or essential characteristics thereof. The foregoing embodiments are therefore to be considered in all respects illustrative rather than limiting on the present teachings described herein. Scope of the present invention is thus indicated by the appended claims rather than by the foregoing description, and all changes that come within the meaning and range of equivalency of the claims are intended to be embraced therein.

1. A system for prevention of infectious diseases in a habitat of animals, said system comprises articles, wherein said articles comprise at least one antimicrobial additive.

2. The system of claim 1 wherein at least one of said articles is a structural part.

3. The system of claim 2, wherein said structural parts selected from the group comprising ceilings, beams, floors, columns, supporters, dividers, panels, grids, lintels, frames, sheds, roofs and fences.

4. The system of claim 1, wherein at least one of said articles is situated within a bedding of a habitat of animals.

5. The system of claim 4, wherein said bedding is composed by said articles.

6. The system of claim 1, wherein said habitat of animals selected from the group consisting of coop, dovecote, house, stable, barn, cote, corral kennel and pond.

7. The system of claim 1, wherein at least one of said articles is placed on a floor.

8. The system of claim 1, wherein at least one of said articles is attached to a floor.

9. The system of claim 1, further comprising articles which comprise at least one insecticide.

10. The system of claim 1, wherein said articles further comprise at least one insecticide.

11. The system of claim 1, wherein said articles are in various shapes.

12. The system of claim 1, wherein said articles are round.

13. The system of claim 1, wherein said articles are selected from the group consisting of: rods, stripes, spheres, cylinders and any combination thereof.

14. The system of claim 1, wherein said articles are sheets or carpets.

15. The system of claim 1, wherein said articles are recyclable.

16. The system of claim 1, wherein said articles are biodegradable.

17. The system of claim 1, wherein at least one of said articles is an auxiliary mean.

18. The system of claim 17, wherein said auxiliary means are selected from the group consisting of: clothes, vehicles and control equipment.

19. The system of claim 17, wherein said auxiliary means are selected from the group consisting of conveyors, piping, hatcheries, incubators, trays, baskets, trolleys, boxes, drinkers, hoppers, silos, tanks, and pans.

20. A method for prevention of infectious diseases in livestock, said method comprising using articles which comprise at least one antimicrobial agent.

21. The method of claim 20, wherein said articles comprise antimicrobial additives.

22. The method of claim 20, said method further comprising using articles which comprise insecticides.

23. The method of claim 20 wherein said method comprising covering coop surfaces with sheets, said sheets comprising antimicrobial additives.

24. The method of claim 20, wherein said articles are auxiliary means.

25. The method of claim 24 wherein said auxiliary means selected from the group consisting of: clothes, vehicles and control equipment.

26. The method of claim 24 wherein said auxiliary means selected from the group consisting of: conveyors, piping, hatcheries, incubators, trays, baskets, trolleys, boxes, drinkers, hoppers, silos, tanks, and pans.
